(19) United States
(12) Patent Application Publication
(10) Pub. No.: US 2011/0059175 A9
(43) Pub. Date: Mar. 10, 2011

(54) ENHANCING SOLUBILITY AND DISSOLUTION RATE OF POORLY SOLUBLE DRUGS

(75) Inventors: Sandrien Janssens, Wilsele (BE); Guy Van Den Mooter, Pellenberg (BE)

(73) Assignee: K.U.LEUVEN RESEARCH AND DEVELOPMENT, B-3000 Leuven (BE)

(21) Appl. No.: 12/296,848

(22) PCT Filed: Apr. 10, 2007
(86) PCT No.: PCT/BE2007/000033

§ 371 Date: Dec. 29, 2008

(30) Foreign Application Priority Data
Apr. 10, 2006 (GB) 0607105.4

(51) Int. Cl. A61K 9/00; A61K 47/30; A61K 31/55; A61K 31/58; A61K 31/175; A61K 31/34; A61K 31/496; A61K 31/426; A61K 31/44; A61K 31/42; A61K 31/505; A61P 31/10

(52) U.S. Cl. 424/486; 514/772.1; 514/772.2; 514/217; 514/176; 514/592; 514/462; 514/254.07; 514/369; 514/356; 514/380; 514/275

(57) ABSTRACT

The present invention relates generally to use of a polyvinyl alcohol-polyethylene glycol graft copolymer (PVA-PEG graft co-polymer), such as Kollicoat IR, in the formulation of solid dispersions of low aqueous solubility and dissolution rate bioactive compound and, more particularly to a system and method for improving the solubility and dissolution rate of such low aqueous solubility and dissolution rate bioactive compound, in particular the drug of low aqueous solubility, such as a BCS Class II or Class IV drug compounds.

### BACKGROUND OF THE INVENTION

A. Field of the Invention

The present invention relates generally to use of a polyvinyl alcohol-polyethylene glycol graft copolymer (PVA-PEG graft co-polymer), such as Kollicoat IR, in the formulation of solid dispersions of low aqueous solubility and dissolution rate bioactive compound and, more particularly to a system and method for improving the solubility and dissolution rate of such low aqueous solubility and dissolution rate bioactive compound, in particular the drug of low aqueous solubility, such as a BCS Class II or Class IV drug compounds.

Several documents are cited throughout the text of this specification. Each of the documents herein (including any manufacturer's specifications, instructions etc.) are hereby incorporated by reference; however, there is no admission that any document cited is indeed prior art of the present invention.

B. Description of the Related Art

Itraconazole, a potent broad-spectrum triazole antifungal drug with activity against histoplasmosis, blastomycosis and onychomycosis (Grant et al., 1989, De Beule and Van Gestel, 2001), is a weak basic compound with an extremely low aqueous solubility (S˜1 ng/mL at neutral pH and S=4 μg/mL at pH 1,). The pKₐ₄ is determined to be 4.0, pKₐ₃ is calculated to be 1.5-2.0, while its other ionisable nitrogens (PKₐ₁ and pKₐ₂) are not protonated between pH 2 and 10. The calculated log P is 6.2 (Peeters et al., 2002). Given the fact that its permeability is adequate, Itraconazole is a class II drug, according to the biopharmaceutical classification system (Amidon et al., 1995).

A severe limitation in the oral bioavailability of class II compounds is that dissolution takes longer than the transit time through their absorptive sites, resulting in incomplete bioavailability (Dressman and Reppas, 2000).

The Biopharmaceutical Classification System (BCS), originally developed by G. Amidon, separates pharmaceuticals for oral administration into four classes depending on their solubility and their absorbability:

### Class I—High Permeability, High Solubility

### Class II—High Permeability, Low Solubility

### Class III—Low Permeability, High Solubility

### Class IV—Low Permeability, Low Solubility

The interest in this classification system stems largely from its application in early drug development and then in the management of product change through its life-cycle. In the early stages of drug development, knowledge of the class of a particular drug is an important factor influencing the decision to continue or stop its development.

The solubility class boundary is based on the highest dose strength of an immediate release (“IR”) formulation and a pH-solubility profile of the test drug in aqueous media with a pH range of 1 to 7.5. Solubility can be measured by the shake-flask or titration method or analysis by a validated stability-indicating assay. A drug substance is considered highly soluble when the highest dose strength is soluble in 250 ml or less of aqueous media over the pH range of 1-7.5. The volume estimate of 250 ml is derived from typical bioequivalence (BE) study protocols that prescribe administration of a drug product to fasting human volunteers with a glass (about 8 ounces) of water. In the absence of evidence suggesting instability in the gastrointestinal tract, a drug is considered highly soluble when 90% or more of an administered dose, based on a mass determination or in comparison to an intravenous reference dose, is dissolved.

Class II drugs are particularly insoluble, or slow to dissolve, but readily are absorbed from solution by the lining of the stomach and/or the intestine. Prolonged exposure to the lining of the GI tract is required to achieve absorption. Such drugs are found in many therapeutic classes. A class of particular interest is antifungal agents, such as itraconazole.

Based on the BCS, low-solubility compounds are compounds whose highest dose is not soluble in 250 mL or less of aqueous media from pH 1.2 to 7.5 at 37° C. See Cynthia K. Brown, et al., “Acceptable Analytical Practices for Dissolution Testing of Poorly Soluble Compounds”, Pharmaceutical Technology (December 2004).

The permeability class boundary is based, directly, on measurements of the rate of mass transfer across human intestinal membrane, and, indirectly, on the extent of absorption (fraction of dose absorbed, not systemic bioavailability) of a drug substance in humans. The extent of absorption in humans is measured using mass-balance pharmacokinetic studies; absolute bioavailability studies; intestinal permeability methods; in vivo intestinal perfusion studies in humans; and in vivo or in situ intestinal perfusion studies in animals. In vitro permeation experiments can be conducted using excised human or animal intestinal tissue and in vitro permeation experiments can be conducted with epithelial cell monolayers. Alternatively, nonhuman systems capable of predicting the extent of drug absorption in humans can be used (e.g., in vitro epithelial cell culture methods). A drug substance is considered highly permeable when the extent of absorption in humans is determined to be greater than 90% of an administered dose, based on mass-balance or in comparison to an intravenous reference dose. A drug substance is considered to have low permeability when the extent of absorption in humans is determined to be less than 90% of an administered dose, based on mass-balance or in comparison to an intravenous reference dose. An IR drug product is considered rapidly dissolving when no less than 85% of the labelled amount of the drug substance dissolves within 30 minutes, using U.S. Pharmacopoeia (USP) Apparatus I at 100 rpm (or Apparatus II at 50 rpm) in a volume of 900 ml or less in each of the following media: (1) 0.1 N HCI or Simulated Gastric Fluid USP without enzymes; (2) a pH 4.5 buffer; and (3) a pH 6.8 buffer or Simulated Intestinal Fluid USP without enzymes.

Many of the known class II drugs are hydrophobic, and have historically been difficult to administer. Moreover, because of the hydrophobicity, there tends to be a significant variation in absorption depending on whether the patient is fed or fasted at the time of taking the drug. This in turn can affect the peak level of serum concentration, making calculation of dosage and dosing regimens more complex. Many of these drugs are also relatively inexpensive, so that simple formulation methods are required and some inefficiency in yield is acceptable.

In the preferred embodiment the drug is itraconazole or a related drug, such as fluoconazole, terconazole, ketoconazole, and saperconazole. Itraconazole is a class II medicine used to treat fungal infections and is effective against a broad spectrum of fungi including dermatophytes (tinea infections), candida, malassezia, and chromoblastomycosis. Itraconazole works by destroying the cell wall and critical enzymes of yeast and other fungal infectious agents. Itraconazole can also decrease testosterone levels, which makes it useful in treating prostate cancer and can reduce the production of excessive adrenal corticosteroid hormones, which makes it useful for Cushing's syndrome. Itraconazole is available in capsule and oral solution form. For fungal infections the recommended dosage of oral capsules is 200-400 mg once a day.

Itraconazole has been available in capsule form since 1992, in oral solution form since 1997, and in an intravenous formulation since 1999. Since Itraconazole is a highly lipophilic compound, it achieves high concentrations in fatty tissues and purulent exudates. However, its penetration into aqueous fluids is very limited. Gastric acidity and food heavily influence the absorption of the oral formulation (Bailey, et al., Pharmacotherapy, 10: 146-153 (1990)). The absorption of itraconazole oral capsule is variable and unpredictable, despite having a bioavailability of 55%.

Other suitable drugs include class II anti-infective drugs, such as griseofulvin and related compounds such as griseoverdin; some anti malaria drugs (e.g. Atovaquone); immune system modulators (e.g cyclosporine); and cardiovascular drugs (e.g. digoxin and spironolactone); and ibuprofen. In addition, sterols or steroids may be used. Drugs such as Danazol, carbamazepine, and acyclovir may also be used in the compositions.

Danazol is derived from ethisterone and is a synthetic steroid. Danazol is designated as 17a-Pregna-2,4-dien-20-yno[2,3-d]-isoxazol-17-ol, has the formula of C₂₂H₂₇NO₂, and a molecular weight of 337.46. Danazol is a synthetic steroid hormone resembling a group of natural hormones (androgens) that are found in the body. Danazol is used in the treatment of endometriosis. It is also useful in the treatment of fibrocystic breast disease and hereditary angioedema. Danazol works to reduce oestrogen levels by inhibiting the production of hormones called gonadotrophins by the pituitary gland. Gonadotrophins normally stimulate the production of sex hormones such as oestrogen and progestogen, which are responsible for body processes such as menstruation and ovulation.

Danazol is administered orally, has a bioavailability that is not directly dose-related, and a half-life of 4-5 hours. Dosage increases in danazol are not proportional to increases in plasma concentrations. It has been shown that doubling the dose may yield only a 30-40% increase in plasma concentration. Danazol peak concentrations occur within 2 hours, but the therapeutic effect usually does not occur for approximately 6-8 weeks after taking daily doses.

Acyclovir is a synthetic nucleoside analogue that acts as an antiviral agent. Acyclovir is available for oral administration in capsule, tablet, and suspension forms. It is a white, crystalline powder designated as 2-amino-1,9-dihydro-9-[(2-hydroxyethoxy)methyl]-6H-purin-6-one, has an empirical formula of C₈H₁₁N₅O₃ and a molecular weight of 225.

Acyclovir has an absolute bioavailability of 20% at a 200 mg dose given every 4 hours, with a half-life of 2.5 to 3.3 hours. In addition, the bioavailability decreases with increasing doses. Despite its low bioavailability, acyclovir is highly specific in its inhibitory activity of viruses due to its high affinity for thymidine kinase (TK) (encoded by the virus). TK converts acyclovir into a nucleotide analogue which prevents replication of viral DNA by inhibition and/or inactivation of the viral DNA polymerase, and through termination of the growing viral DNA chain.

Carbamazepine is used in the treatment of psychomotor epilepsy, and as an adjunct in the treatment of partial epilepsies. It can also relieve or diminish pain that is associated with trigeminal neuralgia. Carbamazepine given as a monotherapy or in combination with lithium or neuroleptics has also been found useful in the treatment of acute mania and the prophylactic treatment of bipolar disorders.

Carbamazepine is a white to off-white powder, is designated as 5H-dibenz[b,f]azepine-5-carboxamide, and has a molecular weight of 236.77. It is practically insoluble in water and soluble in alcohol and acetone. The absorption of carbamazepine is relatively slow, despite a bioavailability of 89% for the tablet form. When taken in a single oral dose, the carbamazepine tablets and chewable tablets yield peak plasma concentrations of unchanged carbamazepine within 4 to 24 hours. The therapeutic range for the steady-state plasma concentration of carbamazepine generally lies between 4 and 10 mcg/mL.

“Class II” drugs of the BCS system dissolve poorly in the gastrointestinal (GI) tract, but are readily absorbed from solution. Such drugs tend to show a significant difference in their eventual absorption, depending on whether the patient is recently fed versus fasting when taking an oral dose. These drugs may also pass through the GI tract with variable proportions of absorption. These effects make oral formulations of Class II drugs both important and difficult.

Thus, there is a need in the art for forms or systems that improve the bioavailability of class II compounds

Three of the parameters that can be manipulated to improve the bioavailability of Class II drugs are (1) particle size, (2) particle dispersion, and (3) release rate. A variety of methods are available for providing drugs in a form which has a large surface, especially as small particles of a few microns in diameter or smaller. Besides fine grinding of crystals, the formation of microparticles from solution by precipitation, spray drying, freeze-drying, and similar methods is known. In addition, the drug solution can be coated onto small particles to achieve its dispersion, as described, for example, in U.S. Pat. No. 5,633,015 to Gilis et al.

Micronized drug on its own tends to re-agglomerate when administered, and this decreases the advantage of improved release kinetics obtained by micronization. Hence, it is also necessary to prevent fine particles of drug from aggregating in formulation. Polymers and other excipients may form a matrix that separates the micronized particles as they are released. Generally, hydrophilic materials, whether polymers or small molecules, are mixed with the fine particles either during or after manufacture. The dried composite materials are typically tableted or put in a capsule. Then, when the capsule or tablet enters the stomach or intestine, the finely dispersed drug is dispersed into the gastrointestinal fluid without aggregating. Such compositions are sometimes referred to as “immediate release”.

Immediate release solid oral dosage forms are typically prepared by blending drug particles with fillers, such as lactose and microcrystalline cellulose; glidants, such as talc and silicon dioxide; disintegrants, such as starch, crosprovidone; and/or lubricants, such as magnesium stearate; and compressing the mixture into the form of a tablet. Alternately the mixture may be filled into a standard capsule, providing a simple oral dosage form.

Hydrophilic polymers may also be used to form a matrix with hydrophobic drugs to separate drug particles, improve wetting and improve dissolution. Polymers such as hydroxylpropylcellulose (HPC), hydroxypropylmethylcellulose (HPMC), and carboxymethylcellulose (CMC) are commonly used for this purpose. The matrix may be formed by blending and direct compression, hot melt extrusion, spray-drying, spray-congealing, wet granulation and extrusion-spheronization.

Although these techniques are effective in the abstract, the rate of absorption is dependant on whether or not the patient ate when taking the drug. For example, the absorption of the drug is significantly higher when the drug is taken with a meal than when it is not. This may be due to competition between dissolution of drug, and aggregation of drug particles as the water-soluble material dissolves. The latter effect may be minimized in the presence of food.

Present invention on the other hand proposes the formulation solid dispersions of class II drugs in a graft copolymer such as PVA-PEG graft copolymer, excipient as Kollicoat IR on the other hand or a like, which resulted in rapid dissolution, with the class II drug, Itraconazole, and supersaturation was maintained for a period of 4 hours for dispersions containing 15, 20 and 25% of Itraconazole. The miscibility of Itraconazole and Kollicoat IR was sufficiently high for drug loads up to 30%.

Though the viscosity of aqueous solutions of Kollicoat IR increases with the polymer concentration, it remains much lower (viscosity of 20% w/w solution is 115 mPas) than that of equivalent solutions of, for instance, cellulose derivatives. Another possible benefit is that Kollicoat IR reduces the surface tension of water (surface tension of a 0% solution is 61.6 mN/m and 41.4 mN/m for a 20% solution) (Kolter et al., 2002, BASF, 2001).

Despite the continuous interest in solid dispersions, the number of different polymeric carriers that have been used during the past 40 years is still rather limited. Indeed, the majority of studies that have been published so far report on the use of polyethylene glycol or polyvinylpyrrolidone. Although combinations of polymers or polymers and surfactants have been proposed in an attempt to tailor the physicochemical properties of the polymeric carriers to those of the dispersed drugs, there is definitely a need to explore new carrier materials (Six et al., 2004, Wang et al., 2005).

In order to contribute in the search of new carriers, we investigated the potential of Kollicoat IR as a polymeric carrier in the formulation of solid dispersions of Itraconazole prepared by hot stage extrusion and found that Kollicoat IR is a valuable excipient in the formulation of dispersed class II compounds and can effectively been used in solid dispersion formulation to increase the solubility and dissolution rate of class II drugs.

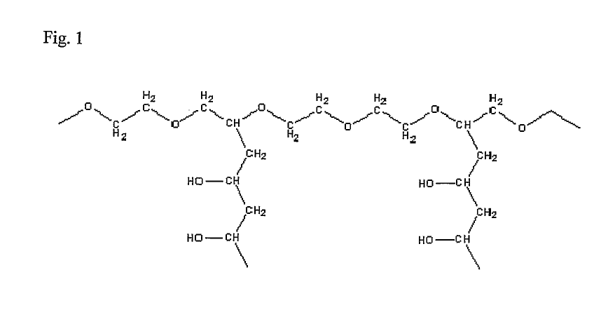

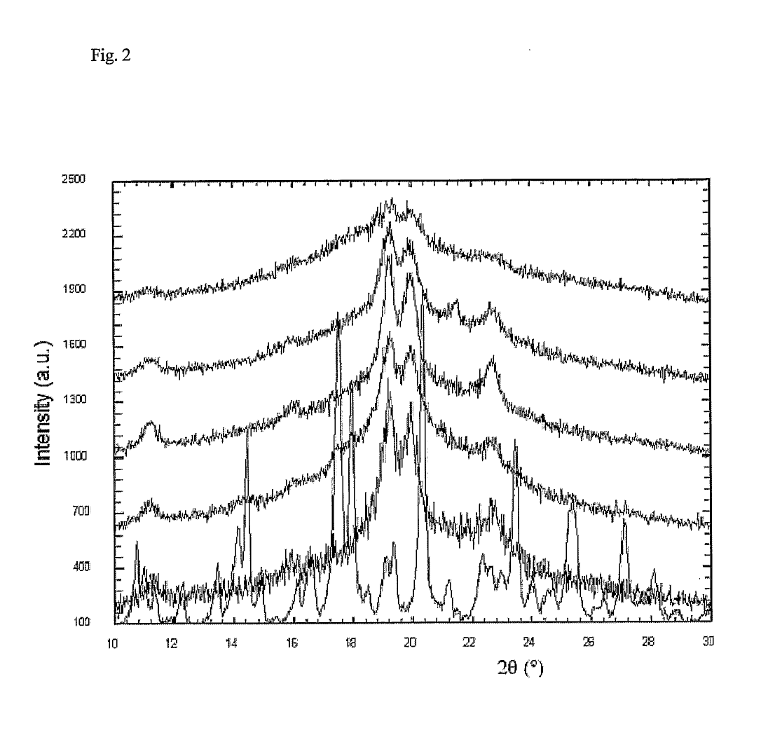

Kollicoat IR, a polyvinyl alcohol-polyethylene glycol graft copolymer, is a pharmaceutical excipient that was especially developed as a coating polymer for instant release tablets. The polyvinyl alcohol moiety has good film-forming properties and the polyethylene glycol part acts as an internal plasticizer. The molecule is hydrophilic and thus readily soluble in water. As its structure (FIG. 1) is non-ionic, its solubility does not change when the pH increases or decreases along the gastro-intestinal tract.

### SUMMARY OF THE INVENTION

The present invention solves the problems of the related art of poor dissolution rate of some of the oral delivered drugs, in particular the class II and the class IV (Biopharmaceutical Classification System) drugs.

We evaluated the graft copolymer, Kollicoat IR, a new pharmaceutical excipient developed as a coating polymer for instant release tablets, as a carrier in solid dispersions of Itraconazole. The solid dispersions were prepared by hot stage extrusion. Hot extrusion can be carried out a temperature of 100-250° C., preferably at 120-220° C., more preferably at 150-200° C. and most preferably at about 180° C.

Modulated temperature differential scanning calorimetry and X-ray powder diffraction were used to evaluate the miscibility of the drug and the carrier. The pharmaceutical performance was evaluated by dissolution experiments, performed in simulated gastric fluid without pepsin (SGFₛₚ). In the X-ray diffractograms no Itraconazole peaks were visible; the polymer on the other hand appeared to be semi-crystalline. Moreover its crystallinity increased during the extrusion process due to exposure to heat and shear forces.

Modulated temperature differential scanning calorimetry analysis showed that the drug and the polymer formed a two phase system. Separate clusters of glassy Itraconazole were present for drug loads of 40% or higher, indicating further phase separation.

Dissolution measurements demonstrated a significantly increased dissolution rate for the solid dispersions compared to physical mixtures. Interestingly the physical mixture made up of glassy Itraconazole and Kollicoat IR (20/80 w/w) showed a dissolution rate and maximum that was much higher than that of the physical mixture made up of crystalline Itraconazole and that of pure glassy Itraconazole.

Present invention demonstrates that a graft copolymer, in particular a polyvinyl alcohol-polyethylene glycol graft copolymer (PVA-PEG graft co-polymer), such as Kollicoat IR is a promising excipient for the formulation of solid dispersions of Itraconazole prepared by hot stage extrusion.

Compared to solid dispersions with separate amorphous drug clusters, our systems of solid dispersions of low aqueous solubility and dissolution rate bioactive compound in graft copolymer (PVA-PEG graft co-polymer), such as Kollicoat IR, demonstrated to be both in terms of dissolution as in terms of stability the solid solutions to be a favourable systems and in particular to have a higher physical stability due to the antiplasticizing effect and protection against recrystallization from the surrounding polymer and to impede aggregation and agglomeration.

In accordance with the purpose of the invention, as embodied and broadly described herein, concerns a medical dosage form of enhanced solubility and dissolution rate in an aqueous environment of low aqueous solubility drugs, characterised in that it comprises a solid dispersion of at least one drug of low aqueous solubility in graft copolymer of 1) water-soluble chains of the vinyl polymer on 2) a polymer chain of water-soluble waxy of alcohols with general formula C₂ₙH₄ₙ+2Oₙ+1 or a polymer chain of polyethylene glycols, polyalkylene glycols, polypropylene glycols, polyisobutylene glycols or polymethylpentene glycols. The graft copolymer has 1) poly(vinyl acetate) and/or poly(vinyl alcohol) and/or poly(vinyl chloride) and poly(vinyl ester) on 2) a polymer chain of polyethylene glycols, polyalkylene glycols, polypropylene glycols, polyisobutylene glycols or polymethylpentene glycols. In a particular embodiment the graft copolymer has a 1) polymer chains of a general structure

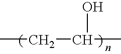

on 2) a polymer chain of the general structure HO—(CH₂—CH₂—O)ₙ—H. Preferably such graft copolymer is non-ionic and reduces the surface tension of water. The solid dispersion is preferably a homogenous dispersion and comprises a supersaturated drug. Most preferably the graft copolymer is Kollicoat IR. Such delivery form of solid dispersions of drug in the graft copolymer can be obtainable after exposure to heat and shear forces during the extrusion process, for instance it can be prepared by hot stage extrusion. But it also can be obtainable by other processes, for instance involving spray-drying.

In one aspect of the invention, the graft copolymer is a graft copolymers of vinyl acetate, crotonic acid and polyalkylene glycol

Another aspect of the invention is that the graft copolymer is a polyvinyl alcohol-polyethylene glycol graft copolymer, in particular such graft polymer may be composed of 75% polyvinyl alcohol units and about 25% polyethylene glycol units with PEG providing the backbone of the branched co-polymer, with the PVA forming the branches

In still another aspect of the invention, the polyethylene glycol graft copolymer has a viscosity lower than 200 mPas in a 20% w/w aqueous solution, preferably the polyethylene glycol graft copolymer has a viscosity lower than 150 mPas in a 20% w/w aqueous solution, more preferably the polyethylene glycol graft copolymer has a viscosity is between 70 mPas and 130 mPas in a 20% W/w aqueous solution, and most preferably polyethylene glycol graft copolymer has a viscosity is about 115 mPas or lower in a 20% w/w aqueous solution.

Another aspect of the invention is that drug in the dosage form of present invention is from the BCS Class II compounds in the Biopharmaceutical Classification System.

Yet another aspect of the invention is that drug in the dosage form of present invention is from the BCS Class IV compounds in the Biopharmaceutical Classification System.

In still another aspect of the invention the medical dosage form comprises a solid dispersion containing up to 40% of drug load, preferably up to 40% of drug load, yet more preferably a drug load in the solid dispersion between 15 to 25%.

The dosage form of present invention is characterised in that it enhances the bioavailability in an aqueous environment of a medically administered bioactive compound, for instance in an aqueous environment such as a gastro-intestinal fluid or a gastric fluid.

The dosage form of present invention can be used to enhance the enhanced solubility and dissolution rate in an aqueous environment of several drugs such as the drugs selected from the group consisting of anti-fungal drugs, antibiotics, steroids, hormones, and immunosuppressants, or the drugs selected from the group consisting of itraconazole, fluoconazole, terconazole, ketoconazole, saperconazole, griseofulvin, griseoverdin, danazole, atovaquone, cyclosporine, digoxin, spironolactone, mefenamic acid, nisoldipine, nifedipine, nicardipine, felodipine, glibenclamide and carbamazepine.

The dosage form of present invention can also be used to enhance the enhanced solubility and dissolution rate in an aqueous environment of several drugs such as the drugs selected from the group consisting of arovaquone, carbamazepine, danazol, glibenclamide, griseofulvin, ketoconazole, troglitazone; or the drug selected from the group consisting of chlorothiazide, furosemide, cyclosporine A, itraconazole; or the drug selected from the group consisting of carbamazepine, dapsone, griseofulvin, buprofen, nifedipine, nitrofurantion, phentytoin, sulfamethoxazole, valproic acid and trimethoprin.

In yet another embodiment of present invention the dosage form of present invention is used to enhance the enhanced solubility and dissolution rate in an aqueous environment of several drugs such as the drugs selected from the group consisting of furosemide, indinavir, ritonavir, saquinavir, acetazolamide and azathioprine; or from the group of compounds consisting of, iopanoic acid, nalidixic acid, nevirapine, praziquantel, rifampicin; or from the group of compounds consisting of albendazole, amitryptyline, artemether, lumefantrine, chloropromazine, ciprofloxacin, clofazimine, efavirenz, lopinavir, folic acid, glibenclamide, haloperidol, ivermectin, mebendazole, niclosamide, pyrantel, pyrimethamine, retinol vitamin, sulfadiazine, sulfasalazine, triclabendazole.

The medical dosage form of present invention may be in the form of a composition elected from the group consisting of tablets, capsules, minitabs, filled tablets, osmotic devices, slurries, dispersions, and suspensions. Moreover the medical dosage form may be particulate. Furthermore the medical dosage form of present invention may comprise a permeation or absorption enhancer or a porous matrix, preferably a molecular sieve.

The drug releasing performance of the medical dosage form of present invention is preferably that 60% of the drug is released in 50 minutes in vitro in an aqueous solution, more preferably that 70% of the drug is released in 50 minutes in vitro in an aqueous solution and most preferably 80% or more of the drug is released in 50 minutes in vitro in an aqueous solution.

Yet another embodiment of present invention is a pharmaceutical composition comprising, the medical dosage form of present invention.

Oral pharmaceutical compositions are preferred for those therapeutic agents that are orally active, and include tablets, capsules, caplets, solutions, suspensions and/or syrups, and may also comprise a plurality of granules, beads, powders or pellets that may or may not be encapsulated. Such pharmaceutical compositions are prepared using conventional methods known to those in the field of pharmaceutical formulation and described in the pertinent texts, e.g., in Remington: The Science and Practice of Pharmacy, 20th Edition, Gennaro, A. R., Ed. (Lippincott, Williams and Wilkins, 2000). Tablets and capsules represent the most convenient oral pharmaceutical compositions, in which case solid pharmaceutical carriers are employed.

Tablets may be manufactured using standard tablet processing procedures and equipment. One method for forming tablets is by direct compression of a powdered, crystalline or granular composition containing the active agent(s), alone or in combination with one or more carriers, additives, or the like. As an alternative to direct compression, tablets can be prepared using wet granulation or dry-granulation processes. Tablets may also be moulded rather than compressed, starting with a moist or otherwise tractable material; however, compression and granulation techniques are preferred.

In addition to the active agent(s), then, tablets prepared for oral administration using the method s of the invention will generally contain other materials such as binders, diluents, lubricants, -disintegrants, fillers, stabilizers, surfactants, colouring agents, and the like. Binders are used to impart cohesive qualities to a tablet, and thus ensure that the tablet remains intact after compression. Suitable binder materials include, but are not limited to, starch (including corn starch and pregelatinised starch), gelatins, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, ethyl cellulose, hydroxyethyl cellulose, and the like), and Veegum. Diluents are typically necessary to increase bulk so that a practical size tablet is ultimately provided.

Suitable diluents include lactose, cellulose, kaolin, mannitol, sodium chloride, dry starch and powdered sugar. Lubricants are used to facilitate tablet manufacture; examples of suitable lubricants include, for example, magnesium stearate and stearic acid. Stearates, if present, preferably present at no more than approximately 2% w/w with respect to the drug-containing core.

Disintegrants are used to facilitate disintegration of the tablet, and are generally starches, clays, celluloses, algins, gums or crosslinked polymers. Fillers include, for example, materials such as silicon dioxide, titanium dioxide, alumina, talc, kaolin, powdered cellulose and microcrystalline cellulose, as well as soluble materials such as mannitol, urea, sucrose, lactose, dextrose, sodium chloride and sorbitol. Stabilisers are used to inhibit or retard drug decomposition reactions that include, by way of example; oxidative reactions. Surfactants may be anionic, cationic, amphoteric or non-ionic surface active agents.

The pharmaceutical composition may also be a capsule, in which case the active agent-containing composition may be encapsulated in the form of a liquid or solid (including particulates such as granules, beads, powders or pellets). Suitable capsules may be either hard or soft, and are generally made of gelatine, starch, or a cellulosic material, with gelatin capsules preferred. Two-piece hard gelatine capsules are preferably sealed, such as with gelatine bands or the like. See, for example, Remington: The Science and Practice of Pharmacy, which describes materials and methods for preparing encapsulated pharmaceuticals. If the active agent-containing composition is present within the capsule in liquid form, a liquid carrier is necessary to dissolve the active agent(s). The carrier must be compatible with the capsule material and all components of the pharmaceutical composition, and must be suitable for ingestion.

Solid pharmaceutical compositions, whether tablets, capsules, caplets, or particulates, may, if desired, be coated so as to provide for delayed release. Pharmaceutical compositions with delayed release coatings may be manufactured using standard coating procedures and equipment. Such procedures are known to those skilled in the art and described in the pertinent texts, e.g., in Remington, supra. Generally, after preparation of the solid pharmaceutical composition, a delayed release coating composition is applied using a coating pan, an airless spray technique, fluidised bed coating equipment, or the like. Delayed release coating compositions comprise a polymeric material, e.g., cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, polymers and copolymers formed from acrylic acid, methacrylic acid, and/or esters thereof. A pharmaceutical preparation. in perorally administrable form containing hydrophobized granules of buflomedil-HCl coated with an acrylic acid polymer and/or a cellulose ether or cellulose ether deriv. to mask the bitter taste of the drug without delaying its release in the digestive tract has for instance been described by Durr Manfred and Gajdos Benedikt in WO9427596.

Alternatively transepidermal effective amounts of drugs may be administered topically on the respective nerve entrapment areas. For such topical treatment the pharmaceutical product can be used as liquid, semi-solid or solid medicine. Liquid medicines are solutions, suspensions, emulsions or dispersions of the above-cited active ingredients or combinations of active ingredients as drops, tinctures and sprays. As semi-solid medicines, for example, gels, ointments, creams and foams are used while, for example, powders, toilet powders, granulates, pellets and microcapsules are used as solid medicines.

A suitable kind of pharmaceutical form may be a topical delivery form of the above-described active ingredient, which is made by the application of the solid, liquid or semi-solid pharmaceutical product onto a gauze strip, a compress or a plaster so that such a gauze strip, such a compress or such a plaster then is only locally applied onto the spot which is to be treated. The pharmaceutical product can be filled into the known receptacles, as for example bottles, tubes, toilet powder boxes and baby powder boxes as well as seal edge bags, which are possibly provided with metering means, as for example droplet forming means, metering valves or metering chambers.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description. It is to be understood that both the foregoing general description and the following detailed description are exemplary and explanatory only and are not restrictive of the invention, as claimed.

### DETAILED DESCRIPTION OF EMBODIMENTS OF THE INVENTION

The following detailed description of the invention refers to the accompanying drawings. The same reference numbers in different drawings identify the same or similar elements. Also, the following detailed description does not limit the invention. Instead, the scope of the invention is defined by the appended claims and equivalents thereof.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

The results of present invention show that a graft co-polymer, more particularly polyvinyl alcohol-polyethylene glycol graft copolymer (for instance Kollicoat IR) is a valuable excipient in the formulation of solid dispersions of a poorly soluble drug such as Itraconazole. A rapid dissolution was obtained and supersaturation was maintained for a period of 4 hours for dispersions containing 15, 20 and 25% of Itraconazole. The miscibility of Itraconazole and Kollicoat IR was sufficiently high for drug loads up to 30%. On the other hand an increase in crystallinity of Kollicoat IR when exposed to heat and shear forces during the extrusion process might be a possible drawback for the of use melting methods to prepare solid dispersions. Good properties are obtainable by controlling shear and heating to achieve temperatures below the re-crystallization temperature or by additives.

The term “graft co-polymer” refers to a copolymer in which chains of a first polymer made of monomer B are grafted onto a second polymer chain of monomer A in other words a graft copolymer has polymer chains of one kind growing out of the sides of polymer chains with a different chemical composition. A preferred graft co-polymer for use in the present invention is a co-polymer consisting of chains of polyvinyl alcohol grafted onto a polyethylene glycol backbone.

The term “supersaturation” is the cause to have a chemical solution to be more highly concentrated than is normally possible under given conditions of temperature and pressure for instance a solution that contains more of the dissolved material than could be dissolved by the solvent under normal circumstances.

A preferred graft co-polymer for improving the solubility and dissolution rate of compound class II compounds by the dosage form of the present invention is a co-polymer being one of polyvinyl alcohol (PVA) and polyethylene glycol (PEG). The PVA-PEG graft co-polymer is available as Kollicoat IR (BASF, Mount Olive, N.J.) or as polyvinyl alcohol/polyethylene glycol graft copolymer (Mowiol GE 597 from Hoechst). The PVA-PEG graft co-polymer consists of 75% polyvinyl alcohol units and 25% polyethylene glycol units with PEG providing the backbone of the branched co-polymer, with the PVA forming the branches. PVA-PEG is very readily soluble in water and has been used mainly for the production of instant-release coatings for tablets.

In present invention involves a dosage form of solid dispersions of low aqueous solubility and dissolution rate bioactive compound in said the graft co-polymer, preferably in a polyvinyl alcohol-polyethylene glycol graft copolymer (PVA-PEG graft co-polymer), such as Kollicoat IR, which has been demonstrated to enhance the solubility and dissolution rate of such low aqueous solubility and dissolution rate bioactive compound.

According to Chiou and Riegelman solid dispersions are defined as ‘a dispersion of one or more active ingredients in an inert carrier or matrix, prepared by the melting, solvent, or melting solvent method (Chiou and Riegelman, 1971). The physical state of the drug in solid dispersions is often transformed from crystalline to amorphous and the dissolution surface increases because of particle size reduction. The presence of the carrier improves the contact between the drug and the dissolution medium and impedes aggregation and agglomeration. The ultimate in particle size reduction are solid solutions in which the drug is molecularly dispersed in the carrier

The extrusion process with this polymer can be optimized by technologies of the state of the art and variation of extend crystallinity will influences the stability and pharmaceutical performance of the extrudates.

The graft co-polymer for use in present invention has preferably the following physical parameters: Molecular weight approx. 45,000 Daltons, pH value of a 20% solution in water of 5.0-8.0, viscosity of a 20% solution in water max. 250 mPa·s (as determined according to EN ISO 2555 at 23° C. using a shear rate of 100 rpm), ester value 10-75.

The polyvinyl alcohol-polyethylene glycol graft copolymer (PVA-PEG graft co-polymer) for use of present invention preferably consists of 75% polyvinyl alcohol units and 25% polyethylene glycol units and also contains approx. 0.3% colloidal silica to improve its flow properties.

Other useful graft copolymers for present invention are the graft copolymers of vinyl acetate, crotonic acid and polyalkylene glycol as described in, for example, German Patent 1,077,430. Their viscosity is preferably of max. 250 mPa·s in a 20% solution in water (as determined according to EN ISO 2555 at 23° C. using a shear rate of 100 rpm)

A particularly preferred copolymer of vinyl acetate, crotonic acid and polyalkylene glycol is a graft copolymer of vinyl acetate, crotonic acid and polyethylene glycol, especially the graft copolymer prepared from 400 parts of vinyl acetate, 32 parts of crotonic acid and 40 parts of 20 polyethylene glycol with a molecular weight of 4,000. Among these copolymers, there may be mentioned the product sold under the name Aristoflex A by Hoechts; its viscosity, in a 5% by weight solution in dimethylformide at 35° C., is 0.0025 to 0.00028 Pa·s.

Graft copolymer in which polyvinyl acetate and/or hydrolysed polyvinyl acetate (polyvinyl alcohol) groups are grafted onto a polyalkylene oxide (preferably polyethylene oxide) backbone. Polymers of this type are described and claimed in EP 219 048B (BASF). These polymers are obtainable by grafting a polyalkylene oxide of molecular weight (number average) 2000-100 000 with vinyl acetate, which may be hydrolysed to an extent of up to 15%, in a weight ratio of polyalkylene oxide to vinyl acetate of 1:0.2 to 1:10. The polyalkylene oxide may contain units of ethylene oxide, propylene oxide and/or butylene oxide; polyethylene oxide is preferred. Preferably the polyalkylene oxide has a number-average molecular weight of from 4000 to 50 000, and the weight ratio of polyalkylene oxide to vinyl acetate is from 1:0.5 to 1:6. Especially preferred are polymers derived from polyethylene oxide of molecular weight 2000-50 000 and having a weight ratio of polyethylene oxide to vinyl acetate of from 1:0.5 to 1:6.

The dosage form may be used for a wide range of low aqueous solubility and dissolution rate active agents or bioactive compounds of the group of ACE inhibitors, adenohypophoseal hormones, adrenergic neuron blocking agents, adrenocortical steroids, inhibitors of the biosynthesis of adrenocortical steroids, alpha-adrenergic agonists, alpha-adrenergic antagonists, selective alpha.sub.2-adrenergic agonists, analgesics, antipyretics and anti-inflammatory agents, androgens, anesthetics, antiaddictive agents, antiandrogens, antiarrhythmic agents, antiasthmatic agents, anticholinergic agents, anticholinesterase agents, anticoagulants, antidiabetic agents, antidiarrheal agents, antidiuretics, antiemetic and prokinetic agents, antiepileptic agents, antiestrogens, antifungal agents, antihypertensive agents, antimicrobial agents, antimigraine agents, antimuscarinic agents, antineoplastic agents, antiparasitic agents, antiparkinsons agents, antiplatelet agents, antiprogestins, antithyroid agents, antitussives, antiviral agents, a typical antidepressants, azaspirodecanediones, barbituates, benzodiazepines, benzothiadiazides, beta-adrenergic agonists, beta-adrenergic antagonists, selective beta.sub.1-adrenergic antagonists, selective beta.sub.2-adrenergic agonists, bile salts, agents affecting volume and composition of body fluids, butyrophenones, agents affecting calcification, calcium channel blockers, cardiovascular drugs, catecholamines and sympathomimetic drugs, cholinergic agonists, cholinesterase reactivators, dermatological agents, diphenylbutylpiperidines, diuretics, ergot alkaloids, estrogens, ganglionic blocking agents, ganglionic stimulating agents, hydantoins, agents for control of gastric acidity and treatment of peptic ulcers, haematopoietic agents, histamines, histamine antagonists, 5-hydroxytryptamine antagonists, drugs for the treatment of hyperlipoproteinemia, hypnotics and sedatives, immunosuppressive agents, laxatives, methylxanthines, monoamine oxidase inhibitors, neuromuscular blocking agents, organic nitrates, opiod analgesics and antagonists, pancreatic enzymes, phenothiazines, progestins, prostaglandins, agents for the treatment of psychiatric disorders, retinoids, sodium channel blockers, agents for spasticity and acute muscle spasms, succinimides, thioxanthines, thrombolytic agents, thyroid agents, tricyclic antidepressants, inhibitors of tubular transport of organic compounds, drugs affecting uterine motility, vasodilators, vitamins and the like, alone or in combination. Although extensive, this list is not intended to be comprehensive.

In another embodiment the dosage form of present invention is used for the poorly soluble drug is selected from the group consisting of carbamazepine, dapsone, griseofulvin, indinavir, nifedipine, nitrofurantion, phentytoin, ritonavir, saquinavir, sulfamethoxazole, valproic acid and trimethoprin.

The dosage form of the present invention can comprises a drug is elected from the group of compounds consisting of acetazolamide, azathioprine, iopanoic acid, nalidixic acid, nevirapine, praziquantel, rifampicin,

In yet an other embodiment of present invention the dosage form comprises a drug selected from the group of compounds consisting of albendazole, amitryptyline, artemether, lumefantrine, chloropromazine, ciprofloxacin, clofazimine, efavirenz, lopinavir, folic acid, glibenclamide, haloperidol, ivermectin, mebendazole, niclosamide, pyrantel, pyrimethamine, retinol vitamin, sulfadiazine, sulfasalazine, triclabendazole.

### EXAMPLES

### Example 1

### Materials

Itraconazole (purity more than 99%) is obtainable from Molekula Ltd Technology House, Old Forge Road, Ferndown Industrial Estate, Wimborne, Dorset, Bh21 7RR, United Kingdom, Phone: +44(0) 1202 863000, Fax: +44(0) 1202 863003, Email: info@molekula.com; ANDAChem, Inc., 6 West Kouzhuang Road, Taiyuan, 030012, People's Republic of China, Phone: 01186-351-734-1915, Fax: 01186-350-202-9235, Email: sales@andachem.com; Sigma-Aldrich, P O Box 14508, St. Louis, Mo., 63178, USA, Phone: 1-800-325-3010, Phone: 1-314-771-5765, Phone: 1-314-771-5750, Fax: 1-800-325-5052, Fax: 1-314-771-5757, Web: http://www.sigma-aldrich.com; RECORDATI S.P.A., Via Civitali 1, Milano, 20148, Italy, Phone: +39 02.48787.1, Fax: +39 02.4870.2322; SK Energy and Chemical, Inc., 22-10 Route 208 South, Fair Lawn, N.J., 07410, USA, Phone: 201-796-4288, Fax: 201-796-3291, b: http://www.skechem.com; eto Corporation, One Hollow Lane, ke Success, N.Y., 11042-1215, USA, Phone: (516) 627-6000, Fax: (516) 627-6093, Email: aceto@aceto.com and others. and Kollicoat IR is a Polyvinyl alcohol-polyethylene glycol graft copolymer, CAS No: 96734-39-3, of a general structure

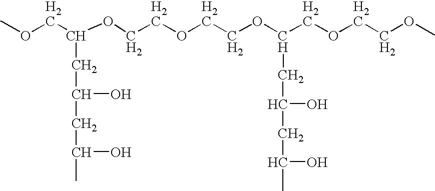

which is obtainable from BASF (Ludwigshafen, Germany).

### Example 2

### Sample Preparation

### 2.1 Hot Stage Extrusion

Hot stage extrusion was performed with a co-rotating, fully intermeshing conical mini twin screw extruder (DSM laboratories, the Netherlands). The temperature was set at 180° C., the screw rate varied from 92 to 100 rpm thermogravimetric analysis showed that no decomposition of the polymer occurred). A load of 5 g per run was fed manually; after feeding the internal circulation time was 5 min. The extrudates were collected after cooling at ambient temperature on a conveyer belt. Extruded samples were subsequently milled for 4 min with a laboratory cutting mill (Kika, Germany) and sieved to exclude particles >355 μm. For two samples the influence of the particle size on the dissolution was investigated. Therefore two fractions were made using sieves of 355, 250 and 90 μm. All samples were stored in a dessicator (P₂O₅) at room temperature and analysed within 3 weeks.

### 2.2 Preparation of Glassy Itraconazole

Glassy Itraconazole was prepared by melting crystalline Itraconazole at 180° C. in an oven and then rapidly cooled to room temperature (Six et al., 2004). The product was subsequently milled and sieved (<355 μm). Glassy Itraconazole was stored in a dessicator (P₂O₅) at room temperature until further analysis (within 3 weeks).

### 2.3 Preparation of Physical Mixtures

Physical mixtures were prepared by mixing Itraconazole and the graft polymer in a mortar for 5 min followed by sieving (<355 μm).

### Example 3

### Characterization of Solid Dispersions

### 3.1 Thermal Analysis

Differential scanning calorimetry (DSC) and Modulated Temperature DSC (MTDSC) measurements were carried out using a Q1000 Modulated DSC (TA Instruments, Leatherhead, UK) equipped with a refrigerated cooling system (RCS). Data were analysed mathematically using Thermal Solutions software (TA Instruments, Leatherhead UK). Dry nitrogen (5.0) at a flow rate of 50 mL/min was used as the purge gas through the DSC cell. TA Instruments (Leatherhead, UK) aluminium open pans were used for all calorimetric studies. The mass of the empty sample pan was matched with the mass of the empty reference pan within ±0.1 mg, the sample mass varied from 13 to 16 mg. The temperature scale and the enthalpic response was calibrated with an Indium standard. The heat capacity signal was calibrated by comparing the response of a sapphire disk with the equivalent literature value at 80° C. Validation of temperature, enthalpy and heat capacity measurement using the same standard materials showed that deviation of the experimental from the reference value was <0.5° C. for the temperature measurement, and <1% for measurement of the heat capacity at 80° C. The amplitude used in the MTDSC experiment was 0.212° C., the period was 40 s, and the underlying heating rate was 2° C./min. The samples were measured from −80° C. to 180° C. The DSC thermogram of Kollicoat IR was recorded with a heating rate of 5° C./min and was measured from 20 to 400° C.

Thermogravimetric analysis was performed with a TGA Q500 (TA-instruments, Leatherhead, UK) using a dry nitrogen purge of 100 ml per min. The 8 mg Kollicoat IR sample was placed in a 100 μl platinum cup and measured from 20 to 400° C. with a heating rate of 5° C. per min.

### 3.2 X-Ray Powder Diffraction Analysis

X-ray powder diffraction was performed at room temperature with a Philips PW diffractometer (beam 173 mm). Monochromatic Cu Kα-radiation (λ=1.5406 Å) was obtained with a Ni-filtration and a system of diverging, receiving and scattering slides of ¼°, 0.2 mm and ¼°, respectively. The diffraction pattern was measured with a voltage of 40 kV and a current of 40 mA in the region of 10°≦2θ≦30° and a step scan mode of 0.02° every 5 seconds. About 200 mg of each sample powder was carefully side-loaded in a sample holder to minimize preferential orientation. The area under the peaks was calculated with WinPlotr.

### 3.3 Dissolution Testing of Solid Dispersions and Physical Mixtures

Dissolution experiments were performed in triplicate on 15/85, 20/80, 25/75, 40/60, and 80/20 Itraconazole/Kollicoat IR (w/w) powdered extrudates and 20/80 Itraconazole/Kollicoat IR (w/w) physical mixtures with either crystalline or glassy Itraconazole. The tests were performed using the USP 24 method 2 (paddle method) in a Hanson SR8plus dissolution apparatus (Chatsworth, Calif.). To simulate the dissolution of a weak basic compound in the stomach, 500 mL of simulated gastric fluid without pepsin (SGF; USP 24) was used as dissolution medium at a temperature of 37° C. and a paddle speed of 100 rpm. Powdered extrudates and physical mixtures (always containing 100 mg of Itraconazole) were added to the dissolution medium. Five-milliliter samples were taken and immediately replaced with fresh dissolution medium at 5, 10, 15, 30, 45, 60, 120, 180, and 240 min. These samples were filtered with 0.45 μm Teflon filters (Macherey-Nagel, Düren, Germany). The filtrates were further analysed by high-performance liquid chromatography (HPLC) (Six et al., 2004).

### 3.4 HPLC Analysis

HPLC analysis was performed with a Merck Hitachi pump L7100, an ultraviolet (UV) detector (L7400), an autosampler (L7200), and an interface (D7000; Merck, Darmstadt, Germany). Acetonitrile/tetrabutyl ammonium hydrogen sulphate 0.01 N (55:45; v/v) was used as mobile phase at a flow rate of 1.0 mL/min, and UV detection at a wavelength of 260 nm. The retention time for Itraconazole was 4.6 min (Six et al., 2004).

### 3.5 Determination of Itraconazole Content in the Solid Dispersions

The solid dispersions were dissolved in dimethylsulfoxide and the Itraconazole content was determined using HPLC.

### Example 4

### Results and Discussion

### 4.1. Physicochemical Analysis

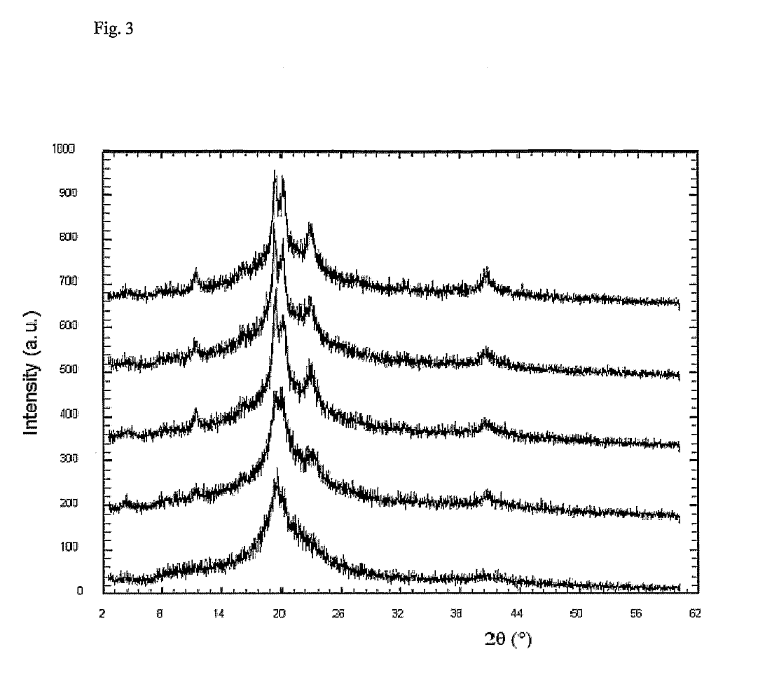

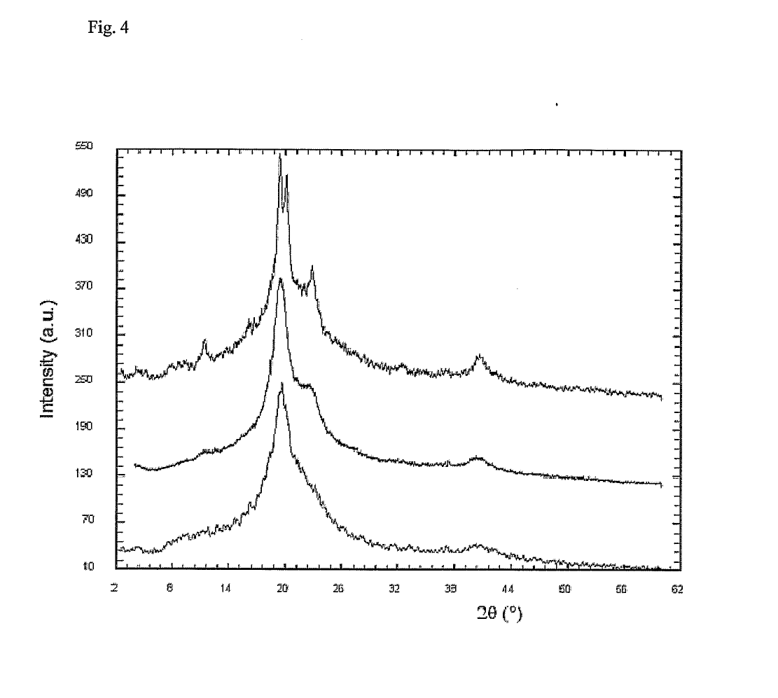

The XRD-spectra of the samples (FIG. 2) were compared with the signal of 100% pure crystalline Itraconazole and that of pure Kollicoat IR that had been extruded. The polymer clearly exhibited a semicrystalline profile. None of the samples showed Itraconazole peaks. Comparison of the spectra of extruded polymer and unprocessed pure polymer revealed that the crystallinity had increased during the extrusion process. Therefore the question arose whether this increase was due to heat exposition or shear forces acting during extrusion. To further investigate this interesting finding the mixing time in the extruder of pure Kollicoat IR extrudates was varied (FIG. 3) and also unprocessed pure Kollicoat IR powder was kept in an oven at 150° C. for 5 min (FIG. 4). The diffractogram of this sample (FIG. 4) proves that even simple heating without applying any other forces on the material causes an increase in crystallinity since a similar diffraction pattern was obtained as for the extruded samples. The peaks of the extrudates (FIG. 3) become more pronounced when the extrusion time is prolonged. The difference in crystallinity between unprocessed Kollicoat IR powder and its extrudate with a 10 min residence time in the extruder was calculated to be 12%.

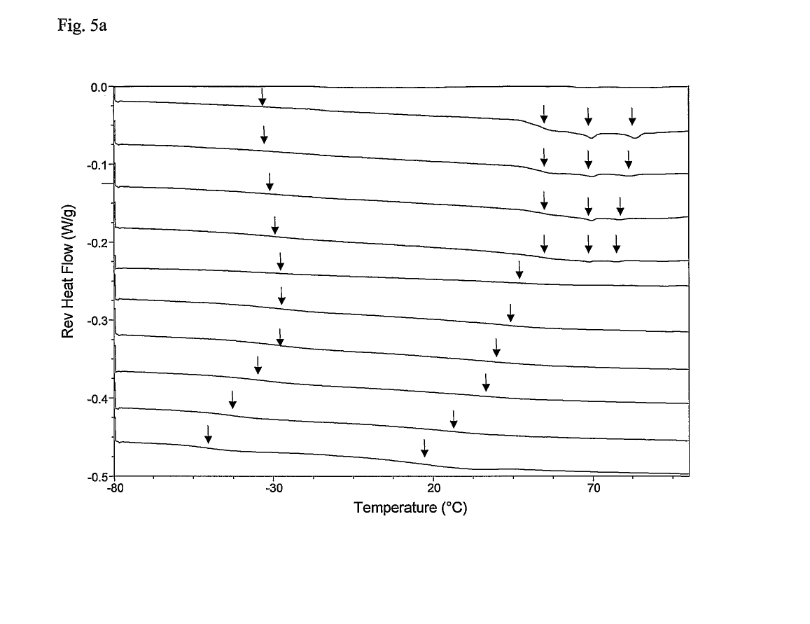

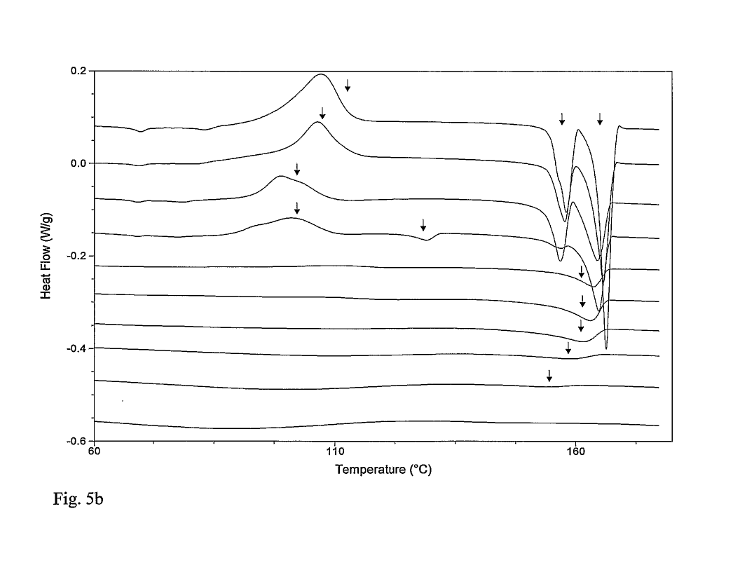

The thermal analysis revealed the complex structure of the solid dispersions (FIGS. 5a, b): two glass transitions were observed for all solid dispersions, an Itraconazole melting endotherm for solid dispersions with a drug load of 10% or more, recrystallization exotherms for solid dispersions with 25% Itraconazole or more, and the additional endotherms around 74 and 90° C. if the Itraconazole concentration is 40% or higher. The presence of two glass transitions indicates the existence of two amorphous phases which are miscible (FIG. 5a). If the concentration of Itraconazole is increased, the highest Tg (Glass Transition Temperature) increases until 40% of drug is reached. From that point on this Tg remains constant around 52±0.4° C., which is slightly below the Tg of pure glassy Itraconazole, which is 59.4° C. (Six et al., 2001). Hence the highest Tg in the solid dispersions is most likely that of a phase containing Itraconazole. Since the Tg remains constant at a value which is just below that of pure glassy Itraconazole, this amorphous phase still contains a small amount of Kollicoat IR which acts as a plasticizer. The first Tg is situated at −52.55° C. when the drug concentration in the solid dispersions is 5% and increases up to −32.5±2° C. This Tg already reaches a constant value from 15% of Itraconazole on. From that point on an endothermic peak at approximately 160° C. is detected which, with increasing drug concentration, increases up to a temperature that corresponds to the melting point of pure Itraconazole. The fact that the first Tg becomes constant while the second one still increases and while an endothermic signal corresponding to the melting of the drug is present, strongly suggests that also the first Tg is that of an amorphous phase containing Itraconazole. The course of the two Tg's point to the fact that the first one is that of a phase which is rich in Kollicoat IR and the second Tg is that of a phase that is rich in Itraconazole. If the concentration of Itraconazole in the solid dispersion is 40% or higher two extra endothermic signals around 74 and 90° C. are appearing. These endothermic transitions can undoubtedly be ascribed to the chiral nematic mesophase of glassy Itraconazole (Six et al., 2001). When Itraconazole is cooled from a melt it remains an isotropic liquid until 90° C. At that temperature a transition from the isotropic liquid to a chiral nematic mesophase occurs and at 74° C. a second transition due to rotational restriction of the molecules is observed. At 59° C. all the mobility of the ordered molecules is frozen at the glass transition. Therefore the presence of these peaks indicates that the mesophase has been formed and hence a separate glassy Itraconazole phase is present.

The melting onset and peak maximum temperature of crystalline Itraconazole (FIG. 5b) decrease as the content of Kollicoat IR increases thus Kollicoat IR acts as an impurity to crystalline Itraconazole. Up to a drug load of 30% (Table 1) no polymorphs are detected but for the 40/60 Itraconazole/Kollicoat IR (w/w) dispersion three melting transitions are present at 124.3° C., 156° C. (peak max.) and 164.7° C. pointing to the presence of polymorfic modifications. As the Itraconazole concentration further increases, only two polymorphic modifications can be detected. Since heating of partially recrystallized pure glassy Itraconazole leads to formation of the most stable polymorph (166.2° C.), the formation of the polymorphs melting at 124.3 and 156° C. are induced by Kollicoat IR (Six et al., 2001). For the samples with a 25% drug load or higher recrystallization exotherms were clearly visible. By subtracting the enthalpy of the recrystallization from the enthalpy of the melting in the total heat flow and dividing this by the enthalpy of fusion of the pure material, an estimation of the amount of crystalline material that was initially present in the sample and not formed during the DSC run could be found (Van den Mooter et al., 2000). However, overlapping of the most stable form with the polymorphic modification, which has a different enthalpy, makes it impossible to calculate the melting enthalpy. Therefore the initial amount of crystalline material can not be calculated in this case.

### 4.2. Dissolution Testing

Milled extrudates with 15, 20, 25, 40 and 80% of Itraconazole were analysed. For two samples, 25/75 and 15/85 Itraconazole/Kollicoat IR w/w, the influence of the particle size on the dissolution was investigated, therefore two fractions were prepared: 355-250 μm and 250-90 μm. These results were compared with the dissolution results of two physical mixtures containing 20% of Itraconazole and 80% of Kollicoat IR, the physical state of Itraconazole in the mixture was either glassy or completely crystalline.

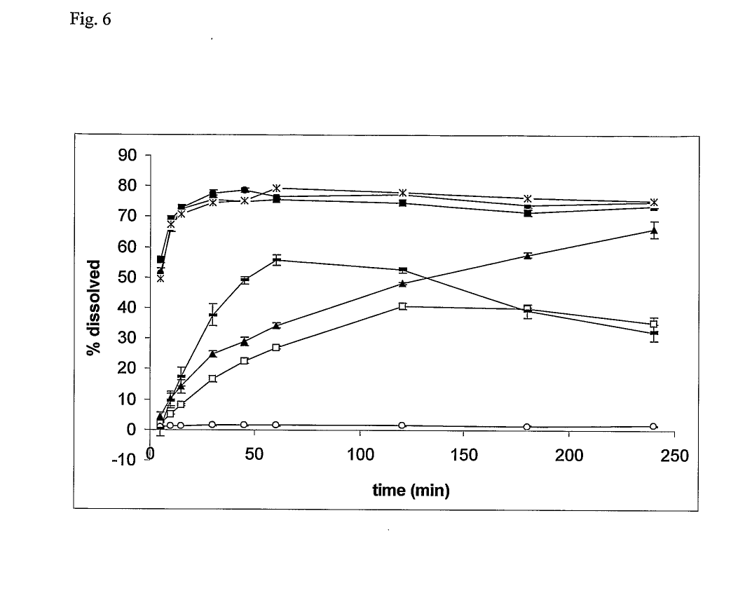

The dissolution profiles (FIG. 6) of the 15/85, 20/80, and 25/75 Itraconazole/Kollicoat IR w/w solid dispersions, size <355 μm, all showed a similar profile. In all three a maximum release of 75% was reached after 30 min and a steady state was maintained during the next 3.5 hours. For the 40/60 Itraconazole/Kollicoat IR w/w sample (FIG. 6) the dissolution rate was remarkably slower, after 4 hours the maximum was still not reached. However, a release of 66% percent was obtained after this time and no precipitation was observed. After 2 hours the 80/20 Itraconazole/Kollicoat IR w/w solid dispersion reaches a maximum release of 40%, after 4 hours only 35% remains in solution due to precipitation (FIG. 6).

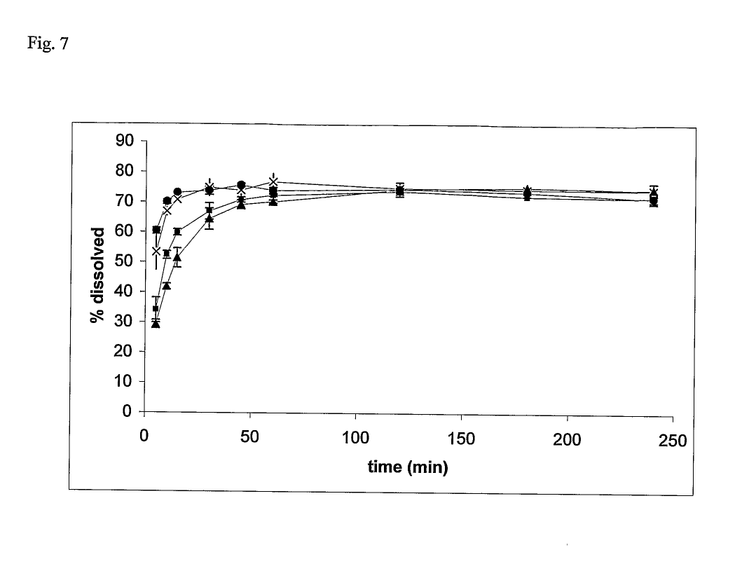

The dissolution profiles of the 355-250 μm and 250-90 μm sized particles of 15/85 and 25/75 Itraconazole/Kollicoat IR w/w (FIG. 7) showed that there is no significant difference in dissolution properties due to the particle size of the milled extrudates.

The two physical mixtures showed a significantly different release. From the physical mixture that was prepared with crystalline Itraconazole a maximum of 1.5% was dissolved after 30 min. The physical mixture that was prepared with glassy Itraconazole showed a release of 55% after 1 hour, which is much higher than the release of pure glassy Itraconazole, which has a release of 14% after 3 hours (Six et al., 2003). Still the maximum release was much lower than for the 20/80 Itraconazole/Kollicoat IR w/w solid dispersion and also the supersaturation could not be maintained.

The explanation for the differences between the dissolution profiles can be found in the different physicochemical properties of the solid dispersions. Based on similarities the analysed samples can be divided into two groups: samples with less than 40% of Itraconazole and samples with 40% or more. The dissolution profile of the 15/85, 20/80, and 25/75 Itraconazole/Kollicoat IR w/w samples was more or less the same. For all these samples it was the case that no separate glassy drug phase was present, the drug and the carrier were mainly present in two separate amorphous phases, although a small crystalline drug fraction was present as well (FIGS. 5a, b). These physicochemical properties are reflected in the dissolution profile that shows that the drug dissolves very fast and remains solubilized in a supersaturated solution. Because of the formation of amorphous phases the dissolution rate is very high since the drug can simply dissolve along with the polymer. Therefore the high aqueous solubility and low viscosity of Kollicoat IR enhances the dissolution process. These properties also explain why the particle size didn't have a significant influence on the dissolution rate. For the 40/60 Itraconazole/Kollicoat IR w/w sample the slower dissolution can be explained by the fact that there is less Kollicoat IR in the dispersion to wet and solubilize the drug, but also because of the separate glassy drug phase (FIG. 5a). In the 80/20 Itraconazole/Kollicoat IR w/w solid dispersion the small amount of Kollicoat IR is not enough to maintain the supersaturation which results in a slight decrease in the dissolution profile due to precipitation. The results of the physical mixture with glassy Itraconazole further confirm that not only a transformation from the drug into the glassy state but also an intensive contact with the carrier is required to have a reasonable dissolution. On the other hand the unusually high dissolution of this physical mixture compared to the dissolution of pure glassy Itraconazole clearly shows that Kollicoat IR influences the saturation solubility of Itraconazole.

### DRAWING DESCRIPTION

### Brief Description of the Drawings

The present invention will become more fully understood from the detailed description given herein below and the accompanying drawings which are given by way of illustration only, and thus are not limitative of the present invention, and wherein:

FIG. 1 provides the chemical structure of Kollicoat IR, a polyvinyl alcohol-polyethylene glycol copolymer.

FIG. 2. demonstrates the overlay of XRPD-spectra of milled Itraconazole/Kollicoat IR extrudates <250 μm with decreasing amount of Itraconazole, 80%, 40%, 20%, 10% to 0%, from top to bottom and 100% pure crystalline Itraconazole (bottom).

FIG. 3. demonstrates the overlay of XRPD-spectra of milled Kollicoat IR extrudates <250 μm and pure untreated Kollicoat (bottom). The mixing times in the extruder decrease from top to bottom, 10, 5, 2, and 0 minutes.

FIG. 4. demonstrates the overlay of XRPD spectra of pure unprocessed Kollicoat IR powder (bottom), pure Kollicoat IR that had been kept in the oven for 5 min at 150° C. (middle), and pure Kollicoat IR that had been extruded (top).

FIG. 5a. demonstrates the reversing heat flow of solid dispersions made up of Itraconazole and Kollicoat IR. From top to bottom: 80%, 60%, 50%, 40%, 30%, 25%, 20%, 15%, 10%, and 5% Itraconazole/Kollicoat IR w/w, all transitions are indicated by arrows.

FIG. 5b. demonstrates the total heat flow of solid dispersions made up of Itraconazole and Kollicoat IR. From top to bottom: 80%, 60%, 50%, 40%, 30%, 25%, 20%, 15%, 10%, and 5% Itraconazole/Kollicoat IR w/w, all transitions are indicated by arrows.

FIG. 6. demonstrates the dissolutions profiles of solid dispersions and physical mixtures: 15/85 Itraconazole/Kollicoat IR w/w solid dispersion; ▪ 20/80 Itraconazole/Kollicoat IR w/w solid dispersion; x 25/75 Itraconazole/Kollicoat IR w/w solid dispersion; ▴ 40/60 Itraconazole/Kollicoat IR w/w solid dispersion; □ 80/20 Itraconazole/Kollicoat IR w/w solid dispersion; ◯ 20/80 crystalline Itraconazole/Kollicoat IR w/w physical mixture; 20/80 glassy Itraconazole/Kollicoat IR w/w physical mixture. Error bars indicate the standard deviation, n=3.

FIG. 7. demonstrates the dissolutions profiles of solid dispersions with particle size between 90-250 or 25-355 μm: x 15/85 Itraconazole/Kollicoat IR w/w solid dispersion, size 355-250 μm; 15/85 Itraconazole/Kollicoat IR w/w, size 250-90 μm; ▴ 25/75 Itraconazole/Kollicoat IR w/w, size 355-250 μm; ▪ 25/75 Itraconazole/Kollicoat IR w/w, size 250-90 μm. Error bars indicate the standard deviation, n=3.

### REFERENCES TO THIS APPLICATION

Grant, S. M., Clissold, S. P., 1989. Itraconazole: a review of its pharmacodynamic and pharmacokinetic properties, and therapeutic use in superficial and systemic mycoses. Drugs 37, 310-344.
De Beule, K., Van Gestel, J., 2001. Pharmacology of itraconazole. Drugs 61, 27-33
Peeters, J., Neeskens, P., Tollenaere, J. P., Van Remoortere P., Brewster, M., 2002. Characterization of the interaction of 2-hydroxypropyl-β-cyclodextrin with itraconazole at pH 2, 4 and 7. J. Pharm. Sci. 91, 1414-1422.
Amidon, G. L., Lennernäs, H., Shah, V. P., Crison, J. R., 1995. Theoretical basis for a biopharmaceutical drug classification; The correlation of in vivo drug product dissolution and in vivo bioavailability. Pharm. Res. 12, 413-420.
Dressman, J. B., Reppas, C., 2000. In vitro-in vivo correlations for lipophilic, poorly water-soluble drugs. Eur. J. Pharm. Sci. 11, S73-S80.
Chiou, W. L., Riegelman, S., 1971. Pharmaceutical applications of solid dispersions. J. Pharm. Sci. 60, 1281-1302.
Leuner, C., Dressman, J. B., 2000. Improving drug solubility for oral delivery using solid dispersions. Eur. J. Pharm. Biopharm. 50, 47-60.
Kolter, K., Gotsche, M., Schneider, T., 2002. Physicochemical characterization of Kollicoat IR. BASF ExAct 8, 2-3.
Kolter, K., 2002. Kollicoat IR—Innovation in instant release film coating. BASF ExAct 8, 4-5.
BASF Aktiengesellschaft, 2001. Technical information, Kollicoat IR.
Six, K., Verreck, G., Peeters, J., Brewster, M., Van den Mooter, G., 2004. Increased physical stability and improved dissolution properties of itraconazole, a class II drug, by solid dispersions that combine fast and slow dissolving polymers. J. Pharm. Sci. 93, 124-131.
Wang, X., Michoel, A., Van den Mooter, G., 2005. Solid state characterisation of ternary solid dispersions composed of PVPVA 64, Myrj 64 and itraconazole. Int. J. Pharm. 303, 54-61.
Six, K., Verreck, G., Peeters, J., Binnemans, K., Berghmans, H., Augustijns, P., Kinget, R., Van den Mooter, G., 2001. Investigation of thermal properties of glassy itraconazole: Identification of a monotropic mesophase. Thermochem. Acta 376, 175-181.
Van den Mooter, G., Wuyts, M., Blaton, N., Busson, R., Grobet, P., Augustijns, P., Kinget, R., 2000. Physical stabilisation of amorphous ketoconazole in solid dispersions with polyvinylpyrolidone K25. Eur. J. Pharm. Sci. 12, 261-269.
Six, K., Berghmans, H., Leuner, C., Dressman, J. B., Van Werde, C., Mullens, J., Benoist, L., Thimon, M., Meublat, L., Verreck, G., Peeters, J., Brewster, M., Van den Mooter, G., 2003. Characterization of solid dispersions of itraconazole and hydroxypropylmethylcellulose prepared by melt extrusion, part II. Pharm. Res. 20, 1047-1054.

1-36. (canceled)

37. A medical dosage form of enhanced solubility and dissolution rate in an aqueous environment of low aqueous solubility drugs, characterised in that it comprises a solid dispersion of at least one low aqueous solubility drug in a graft copolymer of 1) water-soluble chains of a vinyl polymer on 2) a polymer chain of a water-soluble waxy of alcohols with general formula C₂ₙH₄ₙ+2Oₙ+1 or a polymer chain of polyethylene glycols, polyalkylene glycols, polypropylene glycols, polyisobutylene glycols or polymethylpentene glycols.

38. The dosage form of claim 37, wherein the graft copolymer has 1) poly(vinyl alcohol) and/or poly(vinyl chloride) and poly(vinyl ester) on 2) a polymer chain of polyethylene glycols, polyalkylene glycols, polypropylene glycols, polyisobutylene glycols or polymethylpentene glycols.

39. The dosage form of claim 37, wherein the graft copolymer has a 1) polymer chains of a general structure
—(CH₂CHOH)ₙ—
on 2) a polymer chain of the general structure HO—(CH₂—CH₂—O)ₙ—H.

40. The dosage form of claim 37, characterised in that graft copolymer is non-ionic and reduces the surface tension of water.

41. The dosage form of claim 37, characterised in that the graft copolymer is a graft copolymers of vinyl acetate, crotonic acid and polyalkylene glycol.

42. The dosage form of claim 37, characterised in that graft copolymer is a polyvinyl alcohol-polyethylene glycol graft copolymer.

43. The dosage form of claim 40, characterised in that graft copolymer comprises about 75% polyvinyl alcohol units and about 25% polyethylene glycol units with polyethylene glycol units providing the backbone of the branched co-polymer, with the polyvinyl alcohol units forming the branches.

44. The dosage form of claim 37, wherein said low aqueous solubility drug is classifiable as belonging to Class II or class IV of the Biopharmaceutical Classification System.

45. The dosage form of claim 37, wherein the solid dispersion is an homogenous dispersion.

46. The dosage form of claim 37, wherein the low aqueous solubility drug is in a supersaturated solid dispersion.

47. The dosage form of claim 37, wherein the low aqueous solubility drug represents up to 30% of the solid dispersion.

48. The dosage form of claim 47, wherein the low aqueous solubility drug is in a solid dispersion containing between 15 to 25% of drug load.

49. The dosage form of claim 37, wherein the graft copolymer is Kollicoat IR.

50. The dosage form of claim 37, characterised in that the form of solid dispersions of the drug in the graft copolymer is obtainable by exposure to heat and shear forces during the extrusion process.

51. The dosage form of claim 37, characterised in that the form of solid dispersions of drug in the graft copolymer is obtainable by hot stage extrusion.

52. The dosage form of claim 37, characterised in that the form of solid dispersions of drug in the graft copolymer is obtainable by spray-drying.

53. The dosage form of claim 37, further comprising colloidal silica to improve the flow properties of the form.

54. The dosage form of claim 37, wherein the low aqueous solubility drug is selected from the group consisting of arovaquone, carbamazepine, danazol, glibenclamide, griseofulvin, ketoconazole, troglitazone, carbamazepine, dapsone, griseofulvin, buprofen, nifedipine, nitrofurantion, phentytoin, sulfamethoxazole, valproic acid and trimethoprin.

55. The dosage form of claim 37, in a form selected from the group consisting of tablets, capsules, minitabs, filled tablets, osmotic devices, slurries, dispersions and suspensions.

56. The dosage form of claim 37, wherein the low aqueous solubility drug is in the form of particles.

57. The dosage form of claim 37, further comprising a permeation or absorption enhancer.

58. The dosage form of claim 37, further comprising a porous matrix.

59. The dosage form of claim 58, wherein the porous matrix is a molecular sieve.

60. The dosage form of claim 56, wherein the particles are microparticles.

61. A pharmaceutical composition comprising a medical dosage form of enhanced solubility and dissolution rate in an aqueous environment of low aqueous solubility drugs, characterised in that it comprises a solid dispersion of at least one drug of low aqueous solubility in a graft copolymer of 1) water-soluble chains of a vinyl polymer on 2) a polymer chain of water-soluble waxy of alcohols with general formula C₂ₙH₄ₙ+2Oₙ+1 or a polymer chain selected from the group consisting of polyethylene glycols, polyalkylene glycols, polypropylene glycols, polyisobutylene glycols and polymethylpentene glycols.

62. A method for preparing a medical dosage form, characterised in that said method comprises the step of exposing (i) a graft copolymer of 1) water-soluble chains of a vinyl polymer on 2) a polymer chain of water-soluble waxy of alcohols with general formula C₂ₙH₄ₙ+2Oₙ+1 or a polymer chain of polyethylene glycols, polyalkylene glycols, polypropylene glycols, polyisobutylene glycols or polymethylpentene glycols and (ii) a low aqueous solubility drug to an energy input until a solid dispersion is formed of the low aqueous solubility drug as amorphous material entrapped in the graft copolymer.

63. The method according to claim 62, whereby the energy input is heat and/or shear forces.

64. The method according to claim 62, whereby the graft copolymer and the low aqueous solubility drug is exposed to an energy input until an homogenous and supersaturated solid dispersion of the low aqueous solubility drug in the graft copolymer is formed.
